(19) United States
(12) Patent Application Publication
(10) Pub. No.: US 2004/0228824 A1
(43) Pub. Date: Nov. 18, 2004

(54) Cosmetic oil-in-water preparation and use thereof for light skincare

(75) Inventors: Nadine Voigt, (US); Thomas Raschke, (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/785,606

(22) Filed: Feb. 23, 2004

Related U.S. Application Data
(63) Continuation of application No. PCT/EP02/08680, filed on Aug. 3, 2002.

(30) Foreign Application Priority Data
Aug. 23, 2001 (DE) 10141258.4

(51) Int. Cl. A61K 7/06; A61K 7/11

(52) U.S. Cl. 424/70.16

(57) ABSTRACT

The invention is a cosmetic or dermatological preparation designated as a light skincare product of the oil-in-water type, with an emulsifier system, comprising further emulsifiers along with two carbomer thickeners. The invention also includes a method for producing the preparation and using the preparation for cosmetic or medical purposes.

### CROSS-REFERENCE TO RELATED APPLICATIONS

This is a continuation application of PCT/EP02/08680, filed Aug. 3, 2002, which is incorporated herein by reference in its entirety, and also claims the benefit of German Priority Application No. 101 41 258.4, filed Aug. 23, 2001.

### FIELD OF THE INVENTION

The present invention relates to a cosmetic and dermatological preparation referred to as “light skincare product” of the oil-in-water type with an emulsifier system which comprises further emulsifiers as well as two carbomer thickeners, to processes for its preparation, and to its use for cosmetic and medicinal purposes.

### BACKGROUND OF THE INVENTION

The human skin is the largest human organ and performs numerous vital functions. Having an average area of about 2 m² in adults, it has a prominent role as a protective and sensory organ. The purpose of this organ is to transmit and avert mechanical, thermal, actinic, chemical and biological stimuli. In addition, it has an important role as a regulatory and target organ in human metabolism.

The main aim of skincare in the cosmetics sense is to strengthen or restore the skin's natural function as a barrier against environmental influences (e.g. dirt, chemicals, microorganisms) and against the loss of endogenous substances (e.g. water, natural fats, electrolytes), and also to assist its horny layer in its natural regeneration ability in cases of existing damage.

If the barrier properties of the skin are impaired, increased resorption of toxic or allergenic substances or attack by microorganisms may result, leading to toxic or allergic skin reactions.

Another aim of skincare is to compensate for the loss by the skin of sebum and water caused by daily washing. This is particularly important if the natural regeneration ability is inadequate. Furthermore, skincare products should protect against environmental influences, in particular against sun and wind, and delay skin aging.

Medicinal topical compositions usually comprise one or more medicaments in an effective concentration. For the sake of simplicity, in order to clearly distinguish between cosmetic and medicinal use and corresponding products, reference is made to the legal provisions in the Federal Republic of Germany (e.g. Cosmetics Directive, Foods and Drugs Act).

Emulsions are generally understood as meaning heterogeneous systems which consist of two liquids, which are usually referred to as phases, and which are immiscible or miscible with one another only to a limited extent. In an emulsion, one of the two liquids is dispersed in the form of very fine droplets in the other liquid.

If the two liquids are water and oil and oil droplets are very finely dispersed in water, this is an oil-in-water emulsion (O/W emulsion, e.g. milk). The basic character of an O/W emulsion is determined by the water. In the case of a water-in-oil emulsion (W/O emulsion, e.g. butter), the principle is reversed, the basic character being determined here by the oil.

Gel emulsions are sensorily particularly light products with a low content of emulsifiers, structure and/or backbone formers (e.g. fatty alcohols) and lipids. They are characterized by the fact that they can be very readily distributed on the skin and impart a feeling of freshness. Following product application, no or only a little residue should remain on the skin. Gel creams usually comprise a relatively high content of hydrophilic thickeners for thickening and stabilizing the systems, in particular carbomers (polyacrylic acid and derivatives thereof), xanthan gums and other gums, cellulose and derivatives thereof, phyllosilicates, starch and derivatives thereof, polysaccharides, sometimes also special polymers.

Carbomers are polymers with a high molecular weight (>1 mg/mol) consisting of a backbone of polyacrylic acid and small amounts of polyalkenyl-polyether crosslinkages. These water-soluble or dispersible polymers have the property of bringing about a significant increase in the viscosity of the liquid in which they are dissolved or dispersed. This is brought about by the formation of carbomer microgels in water.

A particular class of carbomers are polymeric emulsifiers. Polymeric emulsifiers are mainly polyacrylic acid polymers with a high molecular weight. These emulsifiers have a small lipophilic part in addition to the hydrophilic main part. As a result, they act primarily as emulsifiers, but at the same time also as oil-in-water (O/W) emulsion stabilizers. The lipophilic part adsorbs to the oil phase and the hydrophilic backbone swells in the water phase to form a gel structure around the oil droplets, consequently resulting in stabilization of the emulsion.

Carbomer polymers have been used for more than forty years for the adjustment of rheological properties and structuring in cosmetic products and are referred to as so-called carbomer thickeners.

Since the thickener or the thickener combination is present in the external phase, it has a significant influence on the sensory properties of the product. Customary thickener systems often have the disadvantage that they either cannot be distributed easily, do not convey a feeling of freshness or leave behind an excessively greasy residue on the fingers and/or a harsh, sticky feel on the skin after the product has been distributed on the skin.

Skincare products based on carbomer thickeners in particular have either significant stickiness, insufficient physicochemical stability, poor attractiveness or the use of large amounts of lipids and/or emulsifiers.

The aim was to overcome these disadvantages.

### SUMMARY OF THE INVENTION

Surprisingly, it has been found that cosmetic preparations, in particular in the form of gel emulsions, namely in particular preparations of the O/W emulsion type with a content of hydrocolloids chosen from the group of carbomers of the polyacrylic acid type (i), namely in particular those preparations which are made up as follows:

where at least two carbomers are present in the preparations in individual concentrations of 0.1-0.5% by weight, based on the total weight of the preparations, preferably in total concentrations of 0.2 -0.8% by weight,

where these preparations also comprise one or more lipids (ii),

where the total content of the lipids is chosen from the concentration range from 3 to 18.0% by weight, based on the total weight of the preparations, preferably in total concentrations of from 5 to 15% by weight,

where these preparations also comprise emulsifiers (iii) from the group

glyceryl stearate citrate, and

triceteareth-4 phosphate,

where the total content of the emulsifiers does not exceed 2.0% by weight and is preferably chosen from the concentration range from 0.1 to 1.5% by weight, based on the total weight of the preparations,

where these preparations also comprise at least one fatty alcohol (iv),

where the total content of the fatty alcohols does not exceed 2.0% by weight and is preferably chosen from the concentration range from 0.01 to 1.5% by weight, particularly preferably 0.1 to 1.0% by weight, in each case based on the total weight of the preparations,

where these preparations optionally also comprise,

further hydrocolloids (v) from the group

xanthan gum

where the total content of the further hydrocolloids does not exceed 1.5% by weight and is preferably chosen from the concentration range from 0.1 to 1.0% by weight, based on the total weight of the preparations,

achieve these objects.

It had not been foreseen by the person skilled in the art that the preparations according to the invention

are easier to formulate with sensory additives, such as starch and derivatives thereof, and with linear or cyclic oligo- or polyglucosides

absorb more rapidly into the skin,

are more effective skin-moisturizing preparations,

can be removed more easily from the packaging

better bring to bear the effect of the perfume

are characterized by better care, in particular of greasy skin which is prone to pimples and acne,

are characterized by better care, in particular of normal and combination skin,

are characterized by better care of the skin, in particular of young people

would have better sensory properties, such as, for example, reduced stickiness or a freshening effect on the skin,

have higher stability toward decomposition in oil and water phases and

would be characterized by better dermal compatibility

than the preparations of the prior art.

The preparations according to the invention therefore represent an enrichment of the prior art since as a result of the combination of two chemically different carbomers with a further emulsifier, in particular, the novel type and desired sensory attractiveness is achieved.

### DETAILED DESCRIPTION OF THE PREFERRED EMBODIMENTS

Carbomer is the name for thickeners from the group of crosslinked acrylic acid polymers.

According to the invention, a combination of a nonlinear, crosslinked acrylic acid polymer (referred to below as type I carbomer) and a modified carbomer, namely an acrylic acid/C10-30 alkyl methacrylate copolymer (referred to below as type II carbomer) is used.

Type I carbomers according to the invention have a density of from 0.19 to 0.24 mg/kg (in the dry state) and a viscosity of from 45 000 to 65 000 cP—measured at 25° C. and a concentration of 0.5% by weight in water according to the BF Goodrich method (standard test procedure SA-003).

Type II carbomers according to the invention have a density of 0.19-0.24 mg/kg (in the dry state) and a viscosity of from 1 700 to 15 500 cP—measured at 25° C. and a concentration of 0.2% by weight in water according to the BF Goodrich method (standard test procedure SA-015).

According to the invention, the carbomer(s) of type I can advantageously be chosen, for example, from the Carbopol grades from Goodrich (Carbopol 980, 981, 5984, 2984, Ultrez 10, EDT 2001, EDT 2050) and the carbomer(s) of type ii can, for example, be chosen from the Carbopol grades from Goodrich (Carbopol 1382, EDT 2020, Pemulen TR1 or TR2).

Also advantageous are derivatives of polyacrylic acid, e.g. polymethacrylate, polyglyceryl methacrylate, polyacryidimethyltauramides or polyacrylamide.

Within the scope of the present disclosure, the expression “lipids” is sometimes used as a generic term for fats, oils, waxes and the like, as is entirely familiar to the person skilled in the art. The terms “oil phase” and “lipid phase” are also used synonymously.

For the definition of the lipid phase according to claim 1, it is important that fatty alcohols and emulsifiers are explicitly allocated not to the group of lipids or to the fatty phase of the gel cream, but only to the groups listed below!

Advantageously, the lipid or lipids are chosen from the group of moderately polar to nonpolar lipids. It is preferred to make the proportion by weight of polar lipids in the lipid phase less than about 30%.

Oils and fats differ from one another, inter alia, in their polarity, which is difficult to define. It has already been proposed to adopt the interfacial tension toward water as a measure of the polarity index of an oil or of an oil phase. This means that the lower the interfacial tension between this oil phase and water, the greater the polarity of the oil phase in question. According to the invention, the interfacial tension is regarded as one possible measure of the polarity of a given oil component.

The interfacial tension is the force which acts on an imaginary line of one meter in length in the interface between two phases. The physical unit for this interfacial tension is conventionally calculated from the force/length relationship and is usually expressed in mN/m (millinewtons divided by meter). It has a positive sign if it endeavors to reduce the interface. In the converse case, it has a negative sign.

Table 1 below lists moderately polar lipids which are advantageous according to the invention as individual substances or else as mixtures with one another. The relevant interfacial tensions toward water are given in the last column. It is also advantageous in some circumstances to use mixtures of higher and lower polarity and the like, particularly if the overall polarity of the oil phase corresponds to that of a moderate or low polarity.

TABLE 1

| Trade name | INCI name | (mN/m) |
|---|---|---|
| Isofol ® 14 T | Butyl Decanol + Hexyl Decanol + Hexyl | 27.6 |
| | Octanol + Butyl Octanol | |
| Isofol ® 16 | Hexyl Decanol | 24.3 |
| Eutanol ® G | Octyldodecanol | 24.8 |
| Cetiol ® OE | Dicaprylyl Ether | 22.1 |
| Cetiol ® CC | Dicaprylyl Carbonate | 31.7 |
| Miglyol ® 812 | Caprylic/Capric Triglyceride | 21.3 |
| Cegesoft ® C24 | Octyl Palmitate | 23.1 |
| Isopropylstearate | Isopropyl Stearate | 21.9 |
| Estol ® 1540 | Octyl Octanoate | 30.0 |
| EHC | | |
| Finsolv ® TN | C₁₂₋₁₅ Alkyl Benzoate | 21.8 |
| Cetiol ® SN | Cetearyl Isonoanoate | 28.6 |
| Dermofeel ® | Butylene Glycol Caprylate/Caprate | 21.5 |
| BGC | | |
| Trivent ® OCG | Tricaprylin | 20.2 |
| MOD | Octyldodeceyl Myristate | 22.1 |
| Cosmacol ® ETI | Di-C₁₂₋₁₃ Alkyl Tartrate | 29.4 |
| Miglyol ® 829 | Caprylic/Capric Diglyceryl Succinate | 29.5 |
| Prisorine ® | Octyl Isostearate | 29.7 |
| 2036 | | |
| Tegosoft ® SH | Stearyl Heptanoate | 28.7 |
| Abil ® Wax | Cetyl Dimethicone | 25.1 |
| 9840 | | |
| Cetiol ® LC | Coco-Caprylate/Caprate | 24.8 |
| IPP | Isopropyl Palmitate | 22.5 |
| Luvitol ® EHO | Cetearyl Octanoate | 28.6 |
| Cetiol ® 868 | Octyl Stearate | 28.4 |

For the purposes of the present invention, the oil phase can also advantageously comprise substances chosen from the group of esters of saturated and/or unsaturated, branched and/or unbranched alkanecarboxylic acids having a chain length of from 3 to 30 carbon atoms, and saturated and/or unsaturated, branched and/or unbranched alcohols having a chain length of from 3 to 30 carbon atoms and from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, methyl palmitate, isooctyl stearate, isononyl stearate, isononyl isononanoate, 2-ethylhexyl palmitate, 2-ethylhexyl laurate, 2-hexyldecy laurate, 2-hexyldecyl stearate, 2-octyldodecyl palmitate, oleyl oleate, oleyl erucate, erucyl oleate, erucyl erucate and also synthetic, semisynthetic and natural mixtures of such esters, such as, for example, jojoba oil.

The oil phase can also be chosen advantageously from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24 carbon atoms, in particular 12-18 carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

If desired, fatty and/or wax components which are to be used in the oil phase—as secondary constituents in a minor amount—can be chosen from the group of vegetable waxes, animal waxes, mineral waxes and petrochemical waxes. Examples which are favorable according to the invention are candelilla wax, carnauba wax, Japan wax, esparto grass wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresin, ozocerite (earth wax), paraffin waxes and microcrystalline waxes.

Other advantageous fatty and/or wax components are chemically modified waxes and synthetic waxes such as, for example, those obtainable under the trade names Syncrowax HRC (glyceryl tribehenate), Syncrowax HGLC (C₁₆₋₃₆-fatty acid triglyceride) and Syncrowax AW 1C (C₁₈₋₃₆-fatty acid) from CRODA GmbH, and also montan ester waxes, Sasol waxes, hydrogenated jojoba waxes, synthetic or modified beeswaxes (e.g. dimethicone copolyol beeswax and/or C₃₀₋₅₀-alkyl beeswax), poly-alkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Aiso advantageous are certain organosilicon compounds, which have similar physical properties to the specified fatty and/or wax components, such as, for example, stearoxytrimethylsilane.

If desired, the fatty and/or wax components can be present either individually or as a mixture.

Any desired mixtures of such oil and wax components can also be used advantageously for the purposes of the present invention. In some instances, it can also be advantageous to use waxes, for example cetyl palmitate, as the lipid component of the oil phase.

Of the hydrocarbons, paraffin oil, hydrogenated polyolefins (e.g. hydrogenated polyisobutene), squalane and squalene can be used advantageously for the purposes of the present invention.

According to the invention, emulsions which are particularly advantageous are those which are characterized in that the oil phase consists of at least 10% by weight, preferably of more than 20% by weight, of at least one substance chosen from the group consisting of cyclopentasiloxane, Vaseline (petrolatum), paraffin oil and polyolefins, and, of the latter, preference is given to polydecenes.

The oil phase can advantageously additionally have a content of cyclic or linear silicone oils or consist entirely of such oils, although it is preferable to use an additional content of other oil phase components apart from the silicone oil or the silicone oils.

Cyclomethicone (e.g. octamethylcyclotetrasiloxane, cyclopentasiloxane and cyclohexasiloxane) can be used advantageously. However, other silicone oils can also be used advantageously for the purposes of the present invention, for example hexamethylcyclotrisiloxane, polydimethylsiloxane and poly(methylphenylsiloxane):

The preparations present as gel emulsions according to the invention can comprise further emulsifiers. These emulsifiers can advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearates, sorbitan stearates, sucrose stearates)

b) ethoxylated fatty alcohols and fatty acids

c) ethoxylated fatty amines, fatty acid amides, fatty acid alkanolamides

d) alkylphenol polyglycol ethers (e.g. Triton X)

e) sugar derivatives (esters and/or ethers of glucose, sucrose and other sugars; e.g. alkyl polyglycosides, such as polyglyceryl-3 methylglucose distearate, methylglucose sesquistearate)

The anionic emulsifiers include

a) soaps (e.g. sodium stearate)

b) fatty alcohol sulfates

c) mono-, di- and trialkylphosphoric esters and ethoxylates thereof.

The cationic emulsifiers include

a) quaternary ammonium compounds with a long-chain aliphatic radical, e.g. distearyidimonium chloride.

The amphoteric emulsifiers include

a) alkylamininoalkanecarboxylic acids

b) betaines, sulfobetaines

c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:

fatty alcohol ethoxylates,

ethoxylated wool wax alcohols,

polyethylene glycol ethers of the general formula R—O—(—CH₂—CH₂—O—)ₙ—R′,

fatty acid ethoxylates of the general formula R—COO—(—CH₂—CH₂—O—)ₙ—H,

etherified fatty acid ethoxylates of the general formula R—COO—(—CH₂—CH₂—O—)ₙ—R′,

esterified fatty acid ethoxylates of the general formula R—COO—(—CH₂—CH₂—O—)ₙ—C(O)—R′,

polyethylene glycol glycerol fatty acid esters,

ethoxylated sorbitan esters,

cholesterol ethoxylates,

ethoxylated triglycerides,

alkyl ether carboxylic acids of the general formula R—O—(—CH₂—CH₂—O—)ₙ—CH₂—COOH and n are a number from 5 to 30,

polyoxyethylene sorbitol fatty acid esters,

alkyl ether sulfates of the general formula R—O—(—CH₂—CH₂—O—)ₙ—SO₃—H,

fatty alcohol propoxylates of the general formula R—O—(—CH₂—CH(CH₃)—O—)ₙ—H,

polypropylene glycol ethers of the general formula R—O—(—CH₂—CH(CH₃)—O—)ₙ—R′,

propoxylated wool wax alcohols,

etherified fatty acid propoxylates R—COO—(—CH₂—CH(CH₃)—O—)ₙ—R′,

esterified fatty acid propoxylates of the general formula R—COO—(—CH₂—CH(CH₃)—O—)ₙ—C(O)—R′,

fatty acid propoxylates of the general formula R—COO—(—CH₂—CH(CH₃)—O—)ₙ—H,

polypropylene glycol glycerol fatty acid esters,

propoxylated sorbitan esters,

cholesterol propoxylates,

propoxylated triglycerides,

alkyl ether carboxylic acids of the general formula R—O—(—CH₂—CH(CH₃)O—)ₙ—CH₂—COOH,

alkyl ether sulfates or the parent acids of these sulfates of the general formula R—O—(—CH₂—CH(CH₃)—O—)ₙ—SO₃—H,

fatty alcohol ethoxylates/propoxylates of the general formula R—O—Xₙ—Yₘ—H,

polypropylene glycol ethers of the general formula R—O—Xₙ—Yₘ—R′,

etherified fatty acid propoxylates of the general formula R—COO—Xₙ—Yₘ—R′,

fatty acid ethoxylates/propoxylates of the general formula R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly advantageously having HLB values of 14.5-15.5, provided the O/W emulsifiers have saturated radicals R and R′. If the O/W emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), poiyethyiene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-1 9), polyethylene glycol(20) cetylstearyl ether (ceteareth-20).

It is also advantageous to choose the fatty acid ethoxylates from the following group polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth-1-4 sulfate can be used advantageously as aikyi ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

According to the invention, a preferred emulsifier is glyceryl stearate citrate. This is available, for example, under the product names “IMWITOR® 370” from Huls AG and “Axol C 62®” from Goldschmidt AG.

Fatty alcohols are neutral, readily emulsifiable, high-boiling, oily liquids or soft, colorless masses which are virtually insoluble in water.

According to the invention, monohydric alcohols having 8-30 carbon atoms in straight chains, in particular n-octanol, sec-octanol, n-nonyl alcohol, n-decanol, n-undecanol, dodecanol (lauryl alcohol), myristyl alcohol, cetyl alcohol, stearyl alcohol, oleyl alcohol, isooctyl alcohol, isononyl alcohol, isodecyl alcohol, isotridecyl alcohol and isooctadecyl alcohol, cetyl alcohol, stearyl alcohol, carnaubyl alcohol, ceryl alcohol, myricyl alcohol, cetostearyl alcohol; lauryl alcohol, myristyl alcohol, octyldodecanol, oleyl alcohol and stearyl alcohol, can be used.

Further hydrocolloids which can be used according to the invention are xanthan gum and cellulose derivatives.

Also advantageous is the use of derivatized gums, such as, for example, hydroxypropyl guar (Jaguar® HP 8).

The polysaccharides and derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as they are or in modified form, such as, for example, stearylalkonium hectorite.

In addition, silica gels can also be used advantageously.

The polymers include, for example, polyvinyl alcohols, PVP, PVPNA copolymers, polyglycols.

Xanthan gum (CAS No. 11138-66-2), also called Xanthan, is an anionic heteropolysaccharide which is generally formed by fermentation from corn sugar and is isolated as potassium salt. It is produced from Xanthomonas campestris and a number of other species under aerobic conditions with a molecular weight of 2×10⁶ to 24×10⁶. Xanthan gum is formed from a chain having β-1,4-bonded glucose (cellulose) with side chains. The structure of the subgroups consists of glucose, mannose, glucuronic acid, acetate and pyruvate. Xanthan gum is the name of the first microbial anionic heteropolysaccharide. It is produced from Xanthomonas campestris and a number of other species under aerobic conditions with a molecular weight of 2-15 10⁶. Xanthan gum is formed from a chain having β1,4-bonded glucose (cellulose) with side chains. The structure of the subgroups consists of glucose, mannose, glucuronic acid, acetate and pyruvate. The number of pyruvate units determines the viscosity of the xanthan gum. Xanthan gum is produced in two-day batch cultures with a yield of 70-90%, based on carbohydrate used. Here, yields of 25-30 g/l are achieved. After the culture has been killed, work-up is carried out by precipitation with, for example, 2-propanol. Xanthan gum is then dried and ground.

The aqueous phase of the preparations according to the invention in some instances advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ethers, diethylene glycol monomethyl or monoethyl ethers and analogous products, and also alcohols of low carbon number, e.g. ethanol, isopropanol, 1,2-propanediol and glycerol, and, in particular, one or more thickeners which may advantageously be chosen from the group consisting of silicon dioxide, aluminum silicates.

Particularly advantageous preparations are also obtained if antioxidants are used as additives or active ingredients. According to the invention, the preparations advantageously comprise one or more antioxidants. Favorable, but nevertheless optional, antioxidants which may be used are all antioxidants customary or suitable for cosmetic and/or dermatological applications.

The antioxidants are advantageously selected from the group consisting of amino acids (e.g. glycine, histidine, tyrosine, tryptophan) and their derivatives, imidazoles, (e.g. urocanic acid) and their derivatives, peptides, such as D,L-carnosine, D-carnosine, L-carnosine and their derivatives (e.g. anserine), carotenoids, carotenes (e.g. α-carotene, β-carotene, lycopene) and their derivatives, chlorogenic acid and derivatives thereof, lipoic acid and its derivatives (e.g. dihydrolipoic acid), aurothio-glucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), a-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives (in particular ubiquinone Q10), vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and its derivatives, vegetable polyphenols with a logp of 1-3, α-glucosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenomethionine), stilbenes and their derivatives (e.g. stilbene oxide, trans-stilbene oxide), and the derivatives (salts, esters, ethers, sugars, nucleotides, nucleosides, peptides and lipids) of said active ingredients which are suitable according to the invention.

For the purposes of the present invention, oil-soluble antioxidants can be used particularly advantageously.

A surprising property of the present invention is that preparations according to the invention are very good vehicles for cosmetic or dermatological active ingredients into the skin, preferred active ingredients being antioxidants which are able to protect the skin against oxidative stress. Preferred antioxidants are vitamin E and its derivatives and vitamin A and its derivatives.

The amount of antioxidants (one or more compounds) in the preparations is preferably from 0.001 to 30% by weight, particularly preferably 0.05-5% by weight, in particular 0.1-1.0% by weight, based on the total weight of the preparation.

If vitamin E and/or its derivatives are used as the antioxidant or antioxidants, their respective concentrations are advantageously chosen from the range of 0.001 -10% by weight, based on the total weight of the formulation.

If vitamin A or vitamin A derivatives or carotenes or their derivatives are used as the antioxidant or antioxidants, their respective concentrations are advantageously chosen from the range of 0.001-10% by weight, based on the total weight of the formulation.

The person skilled in the art is of course aware that cosmetic preparations are in most cases inconceivable without the customary auxiliaries and additives. The cosmetic and dermatological preparations according to the invention can, accordingly, also comprise cosmetic auxiliaries, as are customarily used in such preparations, for example bodying agents, stabilizers, fillers, preservatives, perfumes, antifoams, dyes, pigments which have a coloring action, thickeners, surface-active substances, emulsifiers, emollients, moisturizers and/or humectants, anti-inflammatory substances, additional active ingredients such as vitamins or proteins, sunscreens, insect repellants, bactericides, virucides, water, salts, antimicrobial, proteolytic or keratolytic substances, medicaments or other customary constituents of a cosmetic or dermatological formulation such as alcohols, polyols, polymers, foam stabilizers, organic solvents or also electrolytes.

The latter can be chosen, for example, from the group of salts containing the following anions: chlorides, also inorganic oxo element anions, of these, in particular, sulfates, carbonates, phosphates, borates and aluminates. Electrolytes based on organic anions are also advantageous, e.g. lactates, acetates, benzoates, propionates, tartrates, citrates, amino acids, ethylenediaminetetraacetic acid and salts thereof and others. Preferred cations of the salts are ammonium, alkylammonium, alkali metal, alkaline earth metal, magnesium, iron or zinc ions. It does not need to be mentioned that only physiologically acceptable electrolytes should be used in cosmetics. Particular preference is given to potassium chloride, sodium chloride, magnesium sulfate, zinc sulfate and mixtures thereof.

Corresponding requirements apply mutatis mutandis to the formulation of medicinal preparations.

The gel emulsions according to the invention can be used as a basis for cosmetic or dermatological formulations. The latter can have the customary composition and be used, for example, for the treatment and care of the skin and/or the hair, as lip care product, as deodorant product and as make-up or make-up remover product in decorative cosmetics or as a sunscreen preparation. For use, the cosmetic and dermatological preparations according to the invention are applied to the skin and/or the hair in a sufficient amount in a manner customary for cosmetics or dermatological compositions.

Accordingly, for the purposes of the present invention, cosmetic or topical dermatological compositions can, depending on their composition, be used, for example, as a skin protection cream, cleansing milk, sunscreen lotion, nourishing cream, day or night cream, etc. In some circumstances it is possible and advantageous to use the compositions according to the invention as a base for pharmaceutical formulations.

The cosmetic or dermatological compositions according to the invention can, for example, be in the form of preparations which can be sprayed from aerosol containers, squeezable bottles or by means of a pump device, or in the form of a liquid composition which can be applied by means of roll-on devices, but also in the form of an emulsion which can be applied from normal bottles and containers.

Suitable propellants for cosmetic or dermatological preparations which can be sprayed from aerosol containers for the purposes of the present invention are the customary known readily volatile, liquefied propellants, for example hydrocarbons (propane, butane, isobutane), which can be used alone or in a mixture with one another. Compressed air is also used advantageously.

The person skilled in the art is of course aware that there are propellants which are non-toxic per se which would be suitable in principle for realizing the present invention in the form of aerosol preparations, but which must nevertheless be avoided because of their unacceptable impact on the environment or other accompanying circumstances, in particular fluorocarbons and chlorofluorocarbons (CFCs).

Also favorable are cosmetic and dermatological preparations which are in the form of a sunscreen. As well as the active ingredient combinations according to the invention, these preferably additionally comprise at least one UV-A filter substance and/or at least one UV-B filter substance and/or at least one inorganic pigment.

For the purposes of the present invention, however, it is also advantageous to provide cosmetic and dermatological preparations whose main purpose is not protection against sunlight, but which nevertheless have a content of UV protectants. Thus, for example, UV-A or UV-B filter substances are usually incorporated into day creams.

UV protectants, like antioxidants and, if desired, preservatives, also effectively protect the preparations themselves against decay.

Preparations according to the invention can advantageously comprise further substances which absorb UV radiation in the UV-B range, the total amount of filter substances being, for example, from 0.1% by weight to 30% by weight, preferably from 0.5 to 10% by weight, in particular from 1.0 to 6.0% by weight, based on the total weight of the preparations, in order to provide cosmetic preparations which protect the hair and/or the skin from the whole region of ultraviolet radiation. They can also be used as sunscreens for the hair or the skin.

If the emulsions according to the invention contain UV-B filter substances, the latter may be oil-soluble or water-soluble. Examples of oil-soluble UV-B filters which are advantageous according to the invention are:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethyl-amino)benzoate amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzo-phenone;

esters of benzalmalonic acid, preferably di(2-ethylhexyl) 4-methoxy-benzalmalonate;

derivatives of 1,3,5-triazine, preferably 2,4,6-trianilino(p-carbo-2′-ethyl-1-hexyloxy)-1,3,5-triazine.

The list of said UV-B filters, which may be used in combination with the active ingredient combinations according to the invention, is of course not intended to be limiting.

It can also be advantageous to use its UV-A filters which have hitherto been customarily present in cosmetic preparations. These substances are preferably derivatives of dibenzoylmethane, in particular 1-(4′-tert-butylphenyl)-3-(4′-methoxypheny)propane-1,3-dione and 1-phenyl-3-(4′-isopropylphenyl)propane-1,3-dione.

In addition, advantageous UVA filters originate from the group of triazines, such as, for example, 2,4-bis{[4-(2-ethylhexyloxy)-2-hydroxy]phenyl}-6-(4-methoxyphenyl)-1,3,5-triazine (trade name Tinosorb® S), and from the group of triazoles, such as, for example, 2,2′-methylenebis[6-2H-benzotriazol-2-yl]-4-(1,1,3,3-tetramethylbutyl)phenol) (trade name Tinosorb® M). An advantageous water-soluble UVA filter is 2′-bis(1,4-phenylene)-1H-benzimidazole-4,6-disulfonic acid sodium salt (trade name Neo Heliopan AP®).

The amounts used for the UVB combination can be used.

Cosmetic and dermatological preparations according to the invention can also comprise inorganic pigments which are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide.

Further constituents which can be used are:

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and their ethers, preferably ethanol, isopropanol, propylene glycol, glycerol, ethylene glycol, ethylene glycol monoethyl or monobutyl ethers, propylene glycol monomethyl, monoethyl or monobutyl ethers, diethylene glycol monomethyl or monoethyl ethers and analogous products.

The examples below are intended to illustrate the present invention without limiting it. The preparation as such is prior art, covered in particular by raw material manufacturer information.

### EXAMPLES

% by weight

(1) Gel cream

| | |
|---|---|
| Glyceryl stearate citrate | 1.50 |
| Cetylstearyl alcohol | 0.50 |
| Caprylic/capric triglyceride | 1.00 |
| Dicaprylyl carbonate | 2.00 |
| Dimethylpolysiloxane, cyclic | 5.00 |
| (cyclomethicones) | |
| Carbomer type I* | 0.20 |
| Carbomer type II** | 0.20 |
| Dimethicone | 1.00 |
| Methyl palmitate | 2.00 |
| Myristyl myristate | 1.00 |
| TiO₂ | 0.5 |
| Ethylhexylcyanodiphenyl acrylate | 3.0 |
| (octocrylene) | |
| Ubiquinone (Q10) | 0.05 |
| Tocopheryl acetate | 1.00 |
| Glycerol | 7.00 |
| Ethanol | 3.00 |
| Dye | q.s. |
| Fillers/additives (distarch phosphate, SiO₂, | |
| BHT, talc, aluminum starch | |
| octenylsuccinate, cyclodextrin) | |
| Perfume | q.s. |
| Preservative | q.s. |
| Water | 100.00 |
| pH = 6.0 | |

(2) Gel cream

| | |
|---|---|
| Glyceryl stearate citrate | 1.00 |
| Cetylstearyl alcohol | 1.00 |
| Caprylic/capric triglyceride | 2.00 |
| Dicaprylyl carbonate | 1.00 |
| Dimethylpolysiloxane, cyclic | 3.00 |
| (cyclomethicones) | |
| Carbomer type I | 0.10 |
| Carbomer type II | 0.20 |
| Dimethicone | 0.50 |
| Jojoba oil | 2.00 |
| Myristyl myristate | 1.00 |
| Ethylhexyl methoxycinnamate | 2.00 |
| TiO₂ | 1.00 |
| Tocopheryl acetate | 0.50 |
| Glycerol | 7.00 |
| Ethanol | 3.00 |
| Dye | q.s. |
| Fillers/additives (distarch phosphate, SiO₂, | |
| BHT, talc, aluminum starch | |
| octenylsuccinate, cyclodextrin) | |
| Perfume | q.s. |
| Preservative | q.s. |
| Water | 100.00 |
| pH = 6.0 | |

(3) Gel cream

| | |
|---|---|
| Glyceryl stearate citrate | 1.25 |
| Cetylstearyl alcohol | 0.75 |
| Caprylic/capric triglyceride | 1.00 |
| Dicaprylyl carbonate | 2.00 |
| Dimethylpolysiloxane, cyclic | 4.00 |
| (cyclomethicones) | |
| Carbomer type I | 0.15 |
| Carbomer type II | 0.25 |
| Dimethicone | 0.75 |
| Jojoba oil | 1.00 |
| Myristyl myristate | 1.00 |
| Ethylhexylmethoxy cinnamate | 2.00 |
| Bis- | 0.25 |
| ethylhexyloxyphenolmethoxyphenyltriazine | |
| Tocopheryl acetate | 0.75 |
| Glycerol | 10.0 |
| Ethanol | 1.00 |
| Dye | q.s. |
| Fillers/additives (distarch phosphate, SiO₂, | |
| BHT, talc, aluminum starch | |
| octenylsuccinate, cyclodextrin) | |
| Perfume | q.s. |
| Preservative | q.s. |
| Water | 100.00 |
| pH = 6.0 | |

(4) Gel cream

| | |
|---|---|
| Triceteareth-4 phosphate | 1.00 |
| Cetylstearyl alcohol | 1.00 |
| Caprylic/capric triglyceride | 1.00 |
| Dicaprylyl carbonate | 1.00 |
| Dimethylpolysiloxane, cyclic | 3.00 |
| (cyclomethicones) | |
| Carbomer type I | 0.10 |
| Carbomer type II | 0.20 |
| Dimethicone | 0.50 |
| Ethylhexyl cocoate | 0.75 |
| Ethylhexyl methoxycinnamate | 2.00 |
| Glycerol | 7.00 |
| Ethanol | 3.00 |
| Tocopheryl acetate | 0.50 |
| Dye | q.s. |
| Fillers/additives (distarch phosphate, SiO₂, | |
| BHT, talc, aluminum starch | |
| octenylsuccinate, cyclodextrin) | |
| Perfume | q.s. |
| Preservative | q.s. |
| Water | 100.00 |
| pH = 6.0 | |

(5) Gel lotion

| | |
|---|---|
| Glyceryl stearate citrate | 1.25 |
| Cetyl stearyl alcohol | 0.50 |
| Octyldodecanol | 2.00 |
| Dicaprylyl carbonate | 1.00 |
| Dimethylpolysiloxane, cyclic | 3.00 |
| (cyclomethicones) | |
| Carbomer type I* | 0.10 |
| Carbomer type II** | 0.10 |
| Dimethicone | 1.00 |
| Hydrogenated polyisobutene | 2.00 |
| Macadamia oil | 1.00 |
| Myristyl myristate | 0.50 |
| Glycerol | 8.00 |
| Dye | q.s. |
| Fillers/additives (distarch phosphate, SiO₂, | |
| BHT, talc, aluminum starch | |
| octenylsuccinate, cyclodextrin) | |
| Perfume | q.s. |
| Preservative | q.s. |
| Water | 100.00 |
| pH = 5.5 | |

*Carbomer type I: crosslinked acrylic acid polymer
**Carbomer type II: modified acrylic acid polymer, in particular acrylic acid/C10-30 alkyl methacrylate copolymer

That which is claimed:

1. A cosmetic or dermatological preparation including an oil-in-water emulsion, comprising:
a) a polyacrylic acid thickener having non-linear, crosslinked polymer chains, a density of from 0.19 to 0.24 Mg/kg in the dry state, and a viscosity of from 45 000 to 65 000 cP measured at 25° C. and at a concentration of 0.5% by weight in water according to BF Goodrich standard test procedure SA-003;
b) an acrylic acid/C10-30 alkyl methacrylate copolymer having a density of 0.19-0.24 mg/kg in the dry state and a viscosity of from 1 700 to 15 500 cP measured at 25° C. and at a concentration of 0.2% by weight in water according to the BF Goodrich standard test procedure SA-015; the total amount of (a) and (b) being 0.2-0.8% by weight based on the total cosmetic preparation,
c) a lipid phase of up to 18.0% by weight based on the total cosmetic preparation;
d) one or more emulsifiers selected from the group consisting of glyceryl stearate citrate and triceteareth-4 phosphate; and
e) one or more fatty alcohols in an amount of 0.01-2.0% by weight based on the total cosmetic preparation.

2. The preparation as claimed in claim 1, wherein (a) is a swollen microgel.

3. The preparation as claimed in claim 1, wherein (b) is a swollen microgel.

4. The preparation as claimed in claim 1, wherein (a) and (b) are in each case present in an amount of 0.05-0.5% by weight based on the total cosmetic preparation.

5. The preparation as claimed in claim 1, wherein the lipid phase (c) is present in an amount from 3 to 18% by weight based on the total cosmetic preparation.

6. The preparation as claimed in claim 1, wherein the lipid phase (c) is present in an amount from 5 to 15% by weight based on the total cosmetic preparation.

7. The preparation as claimed in claim 1, wherein the one or more emulsifiers (d) are present in an amount from 0.01 to 2.0% by weight based on the total cosmetic preparation.

8. The preparation as claimed in claim 1, wherein the one or more emulsifiers (d) are present in an amount from 0.1 to 1.5% by weight based on the total cosmetic preparation.

9. The preparation as claimed in claim 1, wherein the one or more fatty alcohols (e) are present in an amount of from 0.01 to 1.5% by weight based on the total cosmetic preparation.

10. The preparation as claimed in claim 1, wherein the one or more fatty alcohols (e) are present in an amount of from 0.1 to 1.0% by weight based on the total cosmetic preparation.

11. The preparation as claimed in claim 1, further comprising one or more thickeners selected from the group consisting of cellulose derivatives and xanthan gums.

12. The preparation as claimed in claim 11, wherein the one or more thickeners selected from the group consisting of cellulose derivatives and xanthan gums are present in an amount from 0.01 to 1.5% by weight based on the total cosmetic preparation.

13. The preparation as claimed in claim 11, wherein the one or more thickeners selected from the group consisting of cellulose derivatives and xanthan gums are present in an amount from 0.1 to 1.0% by weight based on the total cosmetic preparation.

14. The preparation as claimed in claim 1, further comprising one or more polyacrylic acid derivatives selected from the group consisting of polymethacrylate, polyglyceryl methacrylate, polyacryldimethyltauramides and polyacrylamide.

15. The preparation as claimed in claim 1, further comprising one or more additional emulsifiers selected from the group consisting of nonionic, anionic or cationic emulsifiers in an amount of from 0.1 to 2.0%.

16. The preparation as claimed in claim 1, further comprising one or more cosmetic auxiliaries or active ingredients.

17. A method of treating or caring for skin, comprising applying to the skin a cosmetic or dermatological preparation including an oil-in-water emulsion, comprising:
a) a polyacrylic acid thickener having non-linear, crosslinked polymer chains, a density of from 0.19 to 0.24 Mg/kg in the dry state, and a viscosity of from 45 000 to 65 000 cP measured at 25° C. and at a concentration of 0.5% by weight in water according to BF Goodrich standard test procedure SA-003;
b) an acrylic acid/C10-30 alkyl methacrylate copolymer having a density of 0.19-0.24 mg/kg in the dry state and a viscosity of from 1 700 to 15 500 cP measured at 25° C. and at a concentration of 0.2% by weight in water according to the BF Goodrich standard test procedure SA-015; the total amount of (a) and (b) being 0.2-0.8% by weight based on the total cosmetic preparation,
c) a lipid phase of up to 18.0% by weight based on the total cosmetic preparation;
d) one or more emulsifiers selected from the group consisting of glyceryl stearate citrate and triceteareth-4 phosphate; and
e) one or more fatty alcohols in an amount of 0.01-2.0% by weight based on the total cosmetic preparation.

18. The method as claimed in claim 17, wherein the preparation is applied to the skin as a light skincare product.

19. The method as claimed in claim 17, wherein the preparation is applied to the skin as a cosmetic having low stickiness and high stability.

20. The method as claimed in claim 17, wherein the preparation is applied to the skin for the treatment of oily, blemished or acne-prone skin.

21. The method as claimed in claim 17, wherein the preparation is applied to the skin for face care.

22. The method as claimed in claim 17, wherein the preparation is applied to the skin as a decorative cosmetic.
